(19) United States
(12) Patent Application Publication
(10) Pub. No.: US 2012/0065694 A1
(43) Pub. Date: Mar. 15, 2012

(54) WEDGE SHAPED CATHETER BALLOONS FOR REPAIR OF DAMAGED VERTEBRA

(76) Inventor: Peter M. Simonson, Longboat Key, FL (US)

(21) Appl. No.: 13/158,297

(22) Filed: Jun. 10, 2011

Related U.S. Application Data
(60) Provisional application No. 61/354,024, filed on Jun. 11, 2010.

(51) Int. Cl. A61B 17/56

(52) U.S. Cl. 606/86 R

(57) ABSTRACT

A balloon catheter tube assembly having an outer catheter tube and a concentric inner catheter onto which a generally wedge shaped balloon is attached. The catheter assembly is inserted into a damaged and compressed vertebra. Upon entry, the generally wedge shaped balloon is inflated to create a generally wedge shaped cavity. To create an optimum cavity, the inner catheter is connected to the generally wedge shaped balloon at a favorable position on either the toe or heel of the generally wedge shaped balloon. After the generally wedge shaped cavity is created, the generally wedge shaped balloon is removed and the generally wedge shaped cavity is filled with bone cement.

### CROSS-REFERENCES TO RELATED APPLICATIONS

This application claims priority under 35 USC 119(e) to U.S. Provisional Application No. 61/354,024, filed on Jun. 11, 2010, which is incorporated for herein by reference in its entirety for all purposes.

### FIELD OF INVENTION

The present invention relates to expandable, wedge shaped balloons and accompanying catheters for use in repairing damaged human vertebra.

### BACKGROUND OF THE INVENTION

Balloon catheters are often used in spinal surgeries to correct spinal problems caused by kyphosis (hunch back) or a fractured vertebra. Kyphosis is a pathological curving of the spine caused by a spinal deformity where a number of spinal vertebra lose some or all of their lordotic profile. It is generally the result of degenerative diseases (such as arthritis), developmental problems, osteoporosis, compression fractures and/or trauma.

Osteoporosis is a major cause of vertebra fractures problem afflicting 55% of Americans aged 50 and above. Of these, approximately 80% are women and, of all women over 50, between 35-50% have at least one fractured vertebra. In the United States, 700,000 vertebral fractures from osteoporosis occur annually. To treat such vertebral fractures, a medical procedure known as kyphoplasty is used.

During kyphoplasty, a surgeon drills into the vertebra bone to form a cavity followed by the insertion of a balloon catheter into the cavity. The inflation of the balloon compacts the softer cancellous bone against the inner surface of the harder and rigid cortical wall of the vertebra bone creating a cavity. When the balloon is deflated and removed, the remaining cavity is filled with a fluid-like filling material, such as methylmethacrylate cement or synthetic bone substitute. This cement hardens and provides structural support for the vertebra. Such a procedure restores the vertebra body height and corrects the spinal angular deformity.

A review of the prior art relating to kyphonplasty shows that there are various inflatable devices, catheters and cements used in kyphonplasty. For example, U.S. Pat. Nos. 4,969,888; 5,108,404; 5,972,015; 6,280,456 and; 6,423,083 all disclose apparatus and methods relating to balloon catheters for the fixation of vertebra fractures.

Balloon catheters were first developed for cardiac (heart) applications and are generally deployed in the vasculature region of blood vessels to reach occlusions resulting from plaque buildup along the vascular walls. In these cases, the catheter and the balloon are aligned with the blood vessel. Once the balloon is expanded, the occlusion is opened. These balloons are usually cylindrical or spherical shaped and are attached to the distal end of the catheter. Over the years, the art of angioplasty has been applied to kyphonplasty. Instead of opening occlusions, these cylindrical or spherical shaped balloons are used to create cavities in vertebrae.

Vertebrae interiors however possess complex geometries making it difficult for inflatable devices to acquire a proper alignment. Such complexity is shown in FIG. 1—a top view of the human vertebra 2. The vertebra 2 includes a vertebral body 4 that extends from the anterior (front or chest) to the posterior (back) side. This vertebral body 4 is generally oval and its geometry is arranged about its natural mid-lateral axis 6 and mid-anterior-posterior axis 8. These axes 6 and 8 intersect in the middle region 10 of the vertebral body 4. The vertebral body 4 includes an exterior formed from compact cortical bone 12. This cortical bone 12 encloses an interior material of reticulated cancellous or spongy bone 14. The spinal canal 16 is located on the posterior side of each vertebra 2. In turn, the vertebral arch 18 surrounds the spinal canal 16. Left and right pedicles 20 of the vertebral arch 18 adjoin the vertebral body 4. The spinous process 22 extends from the posterior of the vertebral arch 18, like the left and right transverse processes 24.

FIG. 2 shows how the thoracolumbar spine is divided into three columns. The anterior column A contains the anterior half of the vertebral body 4. The middle column B contains the posterior half of the vertebral body 4 and the posterior column C contains the pedicles 20 and left and right transverse processes 24. There are generally three fracture types—compression, burst and dislocation. When compression fractures occur, the anterior column A fails. These fractures are most common in older patients with osteoporosis and low rate loading injuries. A burst fracture is where both the anterior A and middle column B fail. These fractures result from trauma where neurologic injuries are common with retropulsion of bony fragments. In dislocation fractures, there is failure in all columns A, B, C and are usually caused by shear/translation, flexion/distraction, or flexion rotation. The treatment for such fractures includes stabilization, deformity/pain reduction, distraction/extension decompression and temporary stabilization until fusion matures. Along with rods, hooks and other instrumentation, kyphonplasty can reduce deformity and pain.

The prior art teaches that the insertion of inflatable devices into vertebra bone first requires forming an access portal or hole. There are generally two approaches—a transpedicular and posterolateral approach. The transpedicular approach is shown in FIG. 3. A catheter is inserted into the vertebral body 4 by drilling an access portal through either pedicle 24. A catheter tube 28 with an un-inflated balloon 30 attached around its distal end penetrates either one of the left or right pedicles 24 and reaches the vertebral body at an angle 26 of about 30-45° to the natural mid-lateral axis 6 and mid-anterior-posterior axis 8 (FIGS. 1 and 2). When expanded, the balloon assumes cylindrical shape around the main axis of the catheter tube 28 that is offset from the natural mid-lateral axis 6 and mid-anterior-posterior axis 8 of the vertebral body. In most cases, the transpedicular approach is desirable given that the pedicle is formed of cortical bone surrounding a small cancellous bone. The horizontal diameter of pedicle increases from 7 mm to 1.5 cm with a vertical diameter approximately 1.5 cm.

The posterolateral approach is shown in FIG. 4. In this procedure, a catheter is inserted directly into the vertebral body by drilling an access portal directly into the cortical bone. A catheter tube 32 containing an uninflated balloon 34 around its distal end is shown. When expanded, the balloon expands outward from the medial distal end of the catheter tube 32. A posterolateral approach is less desirable given that the cortical bone is thinner and may have already experienced compression. Furthermore, a posterolateral procedure involves a costotransversectomy where an incision is made along the paraspinous muscles, spanning about four or five ribs. The rib and transverse process are then resected at one to four levels followed by careful retraction of the pleura that expose the vertebral bodies and pedicles. There are also many other anterior approaches but such approaches involve considerable dissection through various parts of the thorax, abdominal and peritoneal sections of the human body.

To fix vertebral body fractures, the prior art shown in FIGS. 3 and 4 is replete with cylindrical, spherical or elliptical inflatable devices. Such cylindrical or spherical shaped balloons run along the same medial axis as their catheters. When substantially inflated, these balloons create cavities that are offset to one lateral side or another. A good example is the Kyphon® Balloon Kyphoplasty sold by Medtronic and described in U.S. Pat. Nos. 4,969,888 and 5,108, 404 by Scholten et al.

Although later prior art describes doughnut, kidney, banana and elliptical shaped balloons (e.g., U.S. Pat. No. 6,423,083 by Relly et al.), it does not teach the shape of a balloon that will optimally support and fit the vertebral body. In many instances, the spherical or cylindrical balloons are too small and have the wrong configuration to allow the hardened bone cement to properly support the spine (see FIGS. 3 & 4). Most of these spherical or cylindrical balloons are designed to expand the inner volume of the vertebra from 30% to 40%. When they do so, they compact only a portion of the vertebra (FIGS. 3 & 4) and may not restore the vertebra body height or correct the spinal angular deformity.

These types of balloons also fail to restore the vertebra body height because spherical or cylindrical cemented cavities make only a single point of contact on the vertebral body surface. In short, they are circles inside a square or cylinder. Furthermore, their cemented cavities are not strong enough or positioned correctly to accomplish the supportive task that they were originally designed to perform—a major shortcoming.

To illustrate this point, FIG. 5A-E illustrates a prior art transpedicular kyphoplasty procedure. First, FIG. 5A is side view of a vertebral body showing the initial insertion of an elliptical balloon catheter into the damaged vertebral body before the balloon is inflated. FIGS. 5B and 5C is a similar view to FIG. 5A but shows inflation of the balloon 36 to form a cavity 38 in the cancellous bone of the vertebral body. FIG. 5D is a view similar to FIG. 5C but shows the initial stage where methyl methacrylate cement is injected into the cavity 38. FIG. 5E is a similar view to FIG. 5D but shows the cavity 40 after methyl methacrylate cement has hardened.

These drawings illustrate how a prior art inflatable balloon is inserted into bone. When their catheters run along the same medial axis as the balloon, these balloons are restrained to their predetermined shape and size. As shown in FIG. 5B, cylindrical balloons 36 expand to their maximum interior volume. As it does so, the balloon 36 expands around the catheter tube 42 axis and compress cancellous bone 14 to form a cavity 38 in FIG. 5C. In most cases, this expansion is offset to all natural axes 6, 8 of the vertebral body illustrated in FIGS. 1 & 2. The formed cavity 38 is also offset to a lateral side and may extend from top to bottom at an angle to middle or intersection of the mid-lateral axis 6 and mid-anterior-posterior axis 8 (FIGS. 1 & 2).

Due to these compromises, the cavity 38 in FIG. 5E when filled with bone cement may not provide optimal support to the actual area of the compression injury, especially if such fractures are in the periphery of the anterior A and/or middle column B shown in FIG. 2. If not in the exact location of the compression fracture, the capacity of the vertebral body to withstand future weight is diminished.

On the other hand, the larger doughnut, kidney, banana and elliptical shaped balloons expand the inner volume of the vertebra from 70% to 90%. If already fractured or collapsed, such compaction applies substantial pressure (from 50 to 300 psi) to the inner surface of the outer cortical bone and may further damage perfectly good and healthy cancellous and/or cortical bone. Furthermore, an over-expanded balloon may also create spinal cord or root injury.

The unequal compaction of cancellous bone by the prior art balloons may also exert unequal interior forces upon the cortical bone making it difficult to elevate or push broken and compressed bone. Such an over-expansion of a fractured or collapsed vertebral body shown in FIG. 5C may displace fractured bone fragments 44 even further. In fact, the prior art reports that there may be leakage of cement 46 into the anterior and posterior columns of the vertebral body (shown in FIG. 5E). The prior art also states that such anterior leakage is stopped by the anterior longitudinal ligament 48—another shortcoming.

Since prior art balloons fail to accurately compact cancellous bone in vertebral bodies, there is a need for a balloon shape that, when inflated, creates a cavity that optimally compacts and supports the vertebra bone when filled.

Furthermore, there are generally two types of catheters described in the prior art. The first catheter type is shown in FIG. 3. In this type, an expandable balloon 30 simply exits the end of a catheter tube. The second type of catheter is shown in FIG. 4 includes an inner catheter tube within an outer catheter tube. The distal end of the inner catheter tube extends beyond the distal end of the outer catheter tube. The outer diameter of the inner catheter tube is smaller than the inner diameter of the outer catheter tube to create a flow passage between the two catheter tubes. The proximal end of the balloon is bonded to the distal end of the outer catheter tube. The distal end of the balloon is bonded to the distal end of the inner catheter tube. An inflation medium (air or liquid) is pushed through the flow passage between the two tubes expanding the balloon. In most cases, the balloon 34 is elastic (i.e., latex). In such configurations, the balloon can only be symmetrical around a catheter tube. This shortcoming contributes to the misalignment of the balloon and its cavity to the actual compression injury.

The prior art also describes more sophisticated catheters. As described in U.S. Pat. No. 4,969,888 to Scholten et al., the implantation of a spherical or cylindrical balloon includes guide pins, cannula, drill stops, drills, irrigation nozzles, injection gun nozzles, etc. Such over-engineering is misplaced since it produces a spherical or cylindrical cavity that may not support the vertebral body it was designed to restore.

The prior art in FIGS. 3 & 5 also shows how the preferred transpedicular approach does not permit parallel or perpendicular alignment of the catheter tube with either the mid-lateral axis 6 and mid-anterior-posterior axis 8 (see, FIGS. 1 & 2) of the vertebral body. The pedicles 24 in FIG. 2 and any catheter drilled through them will be at about a 30-45° angle 26 with the mid anterior-posterior axis 8 of the vertebral body. In brief, any balloon 30, 34 shown in FIGS. 3 & 4 that is symmetrical to its catheter tube will create an offset cavity. Along with a re-designed balloon, there is also a need to re-design a catheter that can deploy a proper aligned balloon at any angle.

In summary, there is a demand in the industry to provide a balloon that creates a cavity inside the vertebral body whose configuration is optimal for supporting that bone. It must properly move the inferior or superior plate of the vertebral body back into place and restore its original height. Both of these objectives must also be achieved without fracturing any more cortical bone wall of the vertebral body or pushing vertebral bone toward the spinal cord. There is also a need to create catheter assemblies that can achieve virtually any desired offset geometry to properly orient such balloons to the particular internal geometry of targeted vertebral bone bodies.

### BRIEF SUMMARY OF THE INVENTION

The present invention provides a generally wedge shaped balloon (e.g., either a triangular or rectangular prism) for use in a balloon catheter. Also provided are a variety of configurations, some of them offset, for the balloon to connect to the catheter as well as concentric inner and outer catheter tubes. Such a wedge shaped balloon with offset catheter can act along a similar axis to that of the vertebral body (e.g., along the natural mid-anterior-posterior axis 8), thereby creating a cavity that, when filled with cement, provides an optimal configuration for supporting and restoring such vertebral body to its original height. Through the use of memory shape alloys, the inner catheter assumes a second axis to the first axis of the outer catheter. It is the second axis that can align prism-like balloon with the natural mid-anterior-posterior axis of the vertebral body.

With the present invention, the wedge shaped balloon employs the mechanics of a wedge. Instead of applying forces in all directions as spherical or cylindrical balloons do, the present invention preferably applies unequal forces. When such an unequal force meets the resistance of the cortical bone of the vertebral body on its base side, it transfers such force to its inclined surface at various degrees and maximally decompresses the vertebral body at its fracture site. With wedge mechanics, cancellous bone compaction diminishes along the inclined slope of the wedge shaped balloon. Such expansion places the appropriate amount of force only to cancellous bone that needs to be elevated thereby pushing the fractured cortical wall back to or near its normal anatomic position.

In another aspect of the invention, the wedge shaped balloon can be rotated right or left or vertically or horizontally inverted to decompress only the injury site. In a preferred embodiment, it can do so because of concentric memory shaped alloy catheter tubes.

With such embodiments, the wedge shaped balloon and offset catheter creates a cavity inside the vertebral body that is optimal for compacting bone. When an optimally positioned cavity is created, it can properly move the inferior or superior plate of the vertebral body back into place and restore its original height. Since it only targets the fractured, depressed or damaged bone, it will not fracture either cortical or cancellous bone wall of the vertebral body any further. When cement is injected into the cavity and hardens, it leaves behind a supportive cement wedge shaped mold so that the capacity of the vertebral body to withstand future weight is increased.

When aligned, the catheter and the balloon of the present invention allows the surgeon the convenience to choose the best surgical approaches to the spinal injury with a geometry that correctly matches the fracture based on the morphology of the targeted bone taken from diagnostic images.

### BRIEF DESCRIPTION OF THE DRAWINGS

FIG. 1 is a top view of a healthy lumbar vertebra, partially cut away.

FIG. 2 is a side view, partially cut away and in section, of the three columns of the thoracolumbar spine.

FIG. 3 is a top view of the lumbar vertebra shown in FIG. 1 with a prior art catheter and balloon deployed by the transpedicular process prior to inflation.

FIG. 4 is a top view of the lumbar vertebra shown in FIG. 2 with a prior art catheter and uninflated balloon deployed by posterolateral process.

FIG. 5A is a prior art schematic side view of a vertebral body showing the initial insertion of an elliptical balloon in the vertebral body before inflation of the balloon.

FIG. 5B is a similar view to FIG. 5A but shows full inflation of the balloon from FIG. 5A to form a cavity in the cancellous bone of the vertebral body.

FIG. 5C is a view similar to FIG. 5B but after the elliptical balloon and the catheter are removed.

FIG. 5D is a view similar to FIG. 5C but shows the initial stage where methyl methacrylate cement is injected into the vertebral body cavity.

FIG. 5E is a similar view to FIG. 5D but shows the vertebral body after methyl methacrylate cement has hardened.

FIG. 6A is a side view of a wedge shaped balloon of the present invention with an angled and offset catheter on its heel side.

FIG. 6B is similar to FIG. 6A but shows the mechanics of its wedge shape.

FIG. 6C is a schematic side view similar to FIG. 6A but shows a thin wedge embodiment.

FIG. 6D is a schematic side view similar to FIG. 6A but shows a wider wedge embodiment.

FIG. 6E is a schematic back view similar to FIG. 6D but shows the offset catheter channel on the opposite side (left versus right).

FIG. 7A is a schematic side view of a lumbar vertebra showing a middle compression break in a collapsed condition with a catheter of the present invention deploying the transpedicular approach.

FIG. 7B is a similar view to FIG. 7A but shows partial inflation of the wedge shaped balloon of the present invention in raising the compressed vertebra.

FIG. 7C is a similar view to FIG. 7B but shows a fully inflated balloon raising and restoring the compressed vertebra.

FIG. 7D is a view similar to FIG. 7C but shows a cavity after removal of the balloon where methyl methacrylate cement is being injected into the vertebral body.

FIG. 7E is a similar view to FIG. 7D but showing the vertebral body after methyl methacrylate cement has hardened and the catheter is removed.

FIG. 8A is a side view of a wedge shaped balloon with an angled and offset catheter channel on its toe side.

FIG. 8B is similar to FIG. 8A but shows the mechanics of its wedge shape.

FIG. 8C is similar to FIG. 8A but shows a thin wedge embodiment.

FIG. 8D is similar to FIG. 8A but shows a wider wedge embodiment.

FIG. 9A is a schematic side view of a lumbar vertebra showing an anterior compression break in a substantially collapsed condition with a catheter device of the present invention deployed by posterolateral approach.

FIG. 9B is a similar view to FIG. 9A but shows the partial inflation of a wedge shaped balloon to create a cavity in the cancellous bone of the vertebral body.

FIG. 9C is a view similar to FIG. 9B but shows a fully restored vertebra body before methyl methacrylate cement is injected into the cavity of the vertebral body.

FIG. 9D is a similar view to FIG. 9C but shows the vertebral body while methyl methacrylate cement is being inserted into the wedge shaped cavity.

FIG. 9E is a similar view to FIG. 9D but showing the vertebral body after methyl methacrylate cement has hardened and the catheter is removed.

FIG. 10A is a side view of a wedge shaped balloon of the present invention with a heel side catheter showing the acute angles desirable to achieve proper anatomic orientation to the inferior vertebral end (bottom) plate.

FIG. 10B is a wedge shaped balloon with a toe side catheter showing the acute angles desirable to achieve proper anatomic orientation to the inferior vertebral end plate.

FIG. 10C is a side view of an inverted wedge shaped balloon with a heel side catheter showing the acute angles possible with the present invention to achieve proper anatomic orientation to the superior vertebral end (top) plate.

FIG. 10D is a side view of an inverted wedge shaped balloon with a toe side catheter showing the acute angles desirable to achieve proper anatomic orientation to the superior vertebral end (top) plate.

FIG. 10E is a side and cut-away view of the concentric inner and outer catheters of present invention.

FIG. 11A is a schematic side view of spinal vertebra showing fractured anterior (side) and inferior (bottom) end plates.

FIG. 11B is similar to FIG. 11A showing a catheter tube body assembly entering the fractured vertebral body at an acute angle by a posterolateral approach

FIG. 12 is a side and cut-away view of the offset catheter of the present invention entering and inflating its balloon inside a fractured and compressed vertebral body.

FIG. 13 is a side and cut-away view of the wedge shaped balloon of the present invention inflating and using wedge mechanics to restore the compressed vertebral body.

FIG. 14 is a side and cut-away view of a wedge shaped cement mold in the vertebral body.

### DETAILED DESCRIPTION OF THE INVENTION

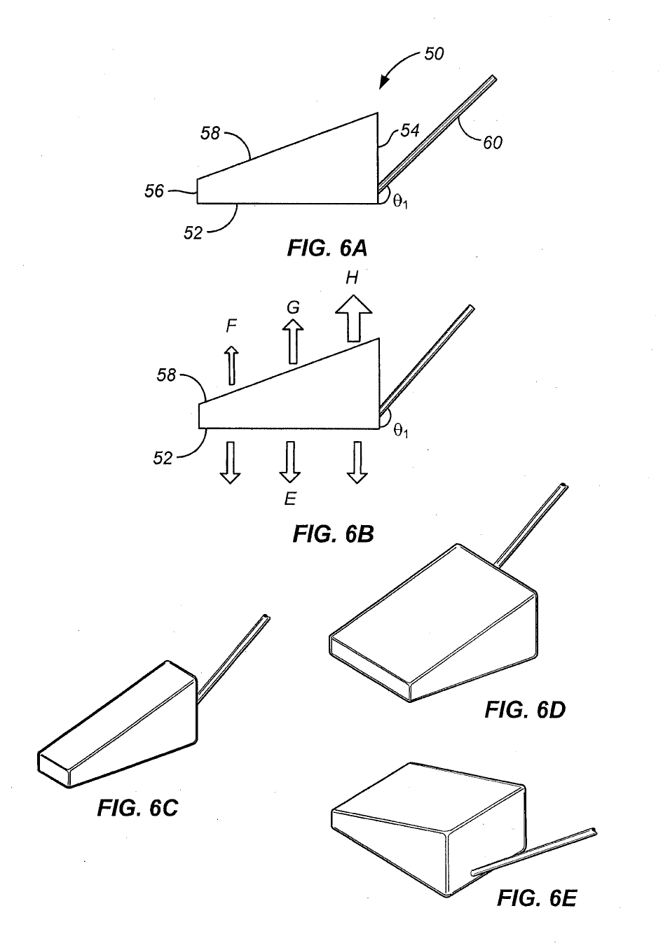

FIGS. 6A-E shows wedge shaped balloons for balloon catheters of the present invention. These wedge shaped can, for example, be either the rectangular prisms shown in FIGS. 6A-E or triangular prisms (i.e., with no toe 56 length). The edges of the wedge shaped balloon may either be sharp as shown in FIGS. 6A-E or rounded. The present invention also preferably includes an offset catheter tube body assembly. In combination, the present invention permits proper cavity formation that is parallel and perpendicular to both the mid-anterior-posterior axis and mid-lateral axis of the vertebral body. Its benefits include the proper decompression of the compression fracture in the appropriate column. When filled with bone cement, such cavity will provide optimal support to the actual area of the compression injury, especially if such fractures are in the periphery of the anterior and/or middle spinal column. Furthermore, a correctly and fully inflated balloon will not damage perfectly healthy and desirous cancellous and/or cortical bone.

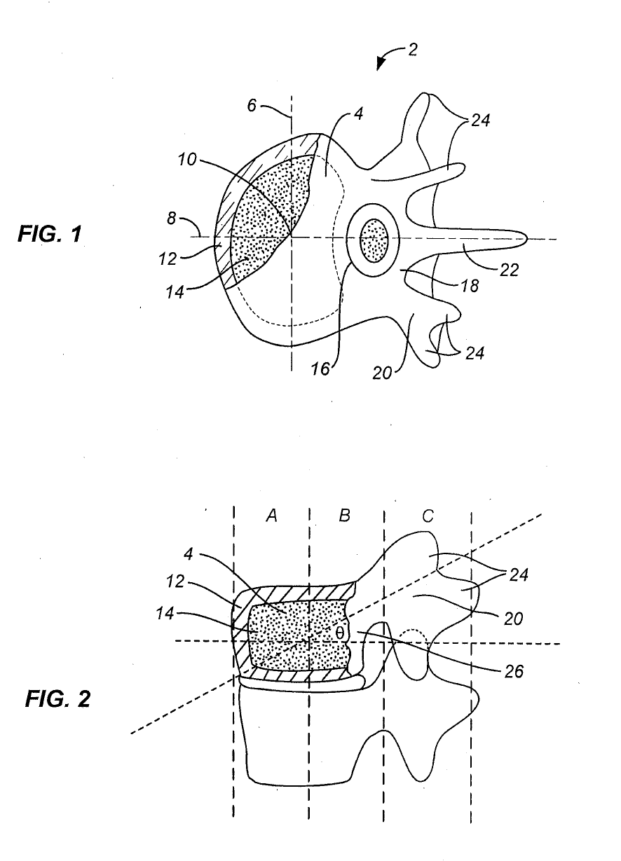

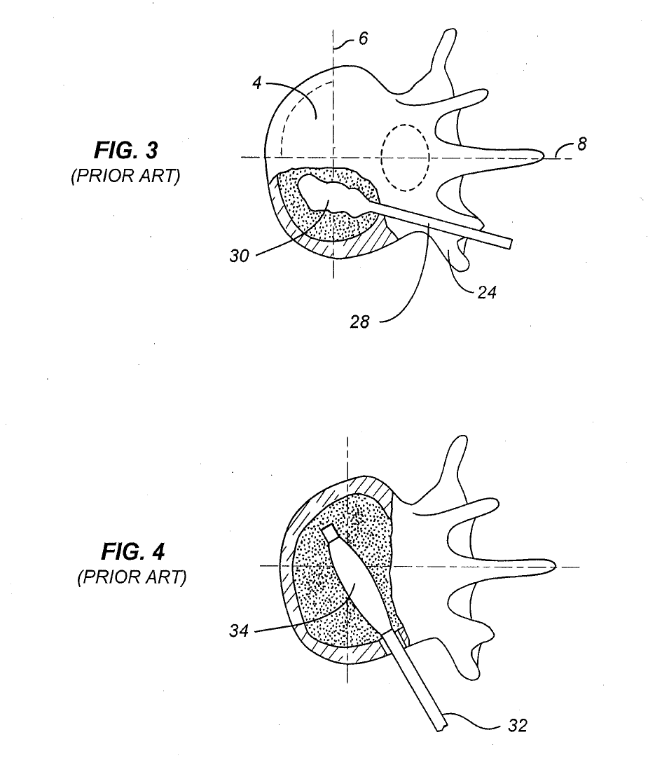

FIG. 6A shows a wedge shaped balloon 50 of the present invention in an inflated condition. Such a wedge shaped balloon 50 has a base side 52, a heel side 54, a toe side 56 and an incline side 58. In FIG. 6A, a catheter tube 60 is attached to the heel side 54 at an offset angle Ø₁. Although the catheter tube may be symmetrical and at a similar angle to the pedicles when expanded, the base 52, heel 54 and toe 56 sides of the wedge shaped balloon is preferably parallel and perpendicular with the mid-lateral axis 6 and mid-anterior-posterior axis 8 (FIGS. 1 & 2) of the vertebral body when inserted.

Some of the advantages of a wedge shaped balloon are illustrated in FIG. 6B. In the classical sense, a wedge functions by converting a force E applied from its base side 52 to its incline side 58. The mechanical advantage (MA) of a wedge is given by the ratio of the length of its slope (S) to its width (W) or MA=S/W. The more acute, or narrow, the angle of a wedge, the greater the ratio of the length of its slope to its width, and thus the mechanical advantage it will yield F<G<H or H>G>F.

FIG. 6C-E shows several embodiments of wedge shaped balloon. The various sizes of a wedge shaped balloon shown in FIGS. 6 C, D and E allows the physician to select a geometry to correctly match the fracture based on morphology of the targeted bone as shown in diagnostic images (i.e., MRJ, CRT). Furthermore, the offset catheter offers surgeons the choice of either the left or right pedicle for insertion (see, FIG. 6E).

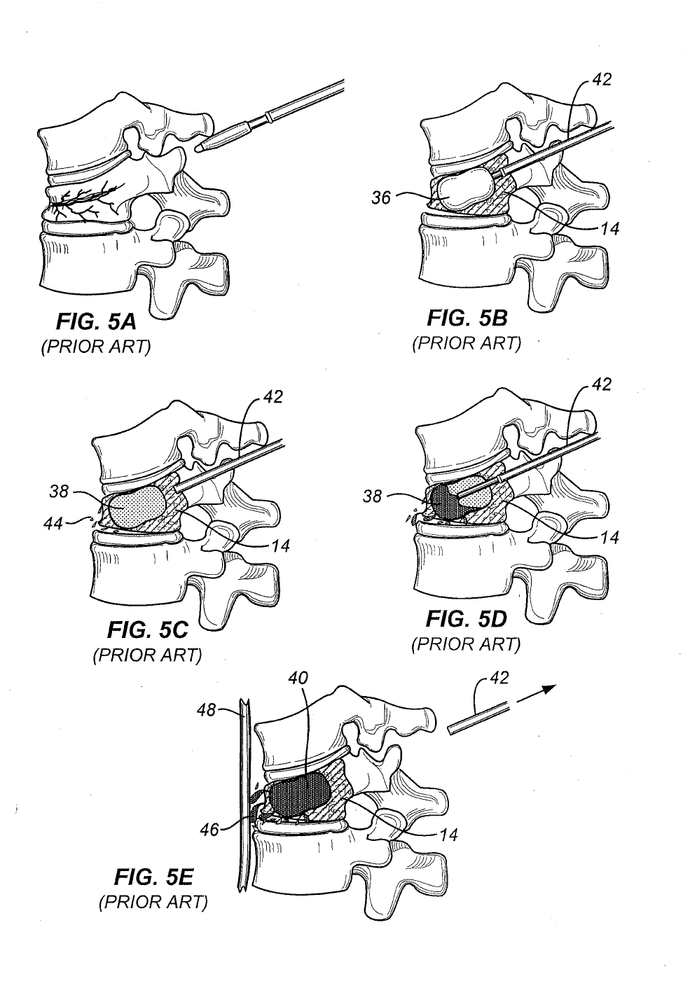

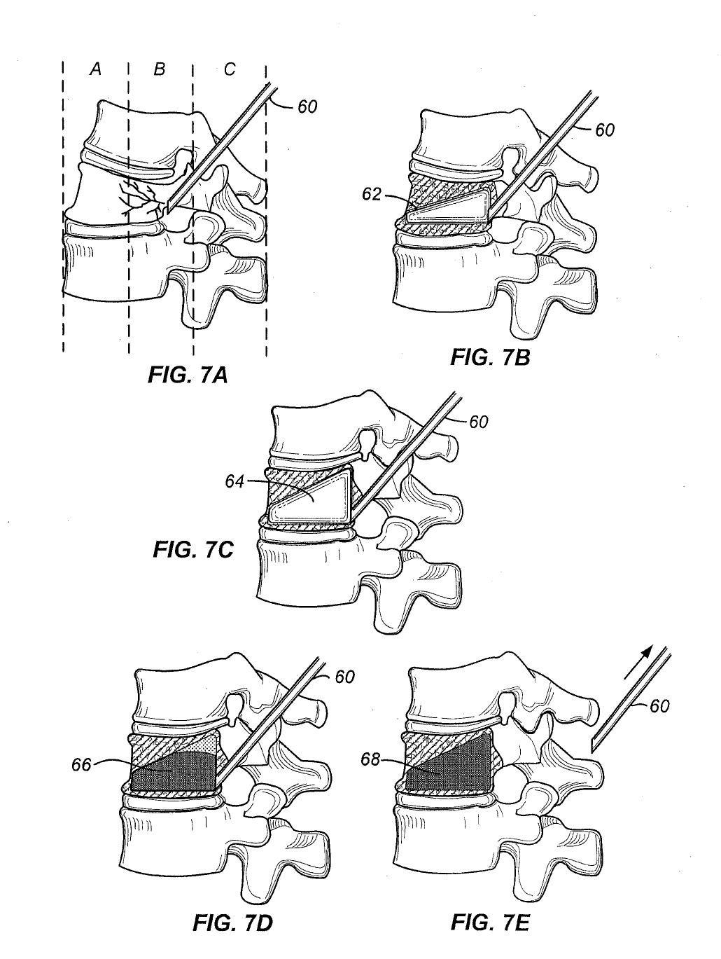

As illustrated in FIGS. 7A-D, the superior mechanics of the wedge can be advantageously applied during the course of spinal surgery. These figures illustrate a transpedicular approach to decompress a middle column B compression fracture. Instead of applying forces in all directions similar to the cylindrical balloon catheter in FIGS. 5A-E, the present invention applies force first onto its base 52 (FIG. 6B). When this force meets the resistance of the inferior end plate (bottom) of the vertebral body, it transfers force upward to its inclined surface at various degrees of force F, G, H (FIG. 6B) and decompresses the vertebral body exactly at its compression fracture site in the middle column B.

Due to the geometry shown in FIGS. 7, maximum cancellous bone compaction does not occur in the entire vertebral body. Instead, maximum cancellous bone compaction occurs at the top H of the incline side 58 of the present invention (FIG. 6B). Furthermore, cancellous bone compaction diminishes from top H to bottom of the inclined slope G, F of the wedge shaped balloon. When complete, the expansion places the appropriate amount of force onto cancellous bone to elevate or push the fractured cortical wall back to or near its normal anatomic position before the fracture occurred. Furthermore, the wedge shaped balloon can be inverted so its base lies parallel with the superior (top) cortical bone of the vertebral body. When it does so, it can transfer its force downward and again decompress the vertebral body based on the morphology of the targeted bone and site of injury.

Referring again to FIGS. 7A-E, FIG. 7A is a schematic side view of a lumbar vertebra showing middle compression break B in a substantially collapsed condition with a catheter 60 deployed by the transpedicular access. FIG. 7B is a similar view to FIG. 7A but shows the partial inflation of the wedge shaped balloon 62, the mechanics of a wedge shifting and applying the decompression force toward the middle compression break B. FIG. 7C is a view similar to FIG. 7B but shows a fully inflated balloon 64 restoring the vertebral body to its original height. FIG. 7D is similar to FIG. 7C but shows how removing the balloon leaves behind a cavity 66 that is being injected with methyl methacrylate cement. Such a wedge shaped cavity 66 when filled with bone cement can provide optimal support to actual area of the compression injury especially if such fractures are in the middle column B (FIG. 7A). FIG. 7E is a similar view to FIG. 7D but shows the vertebral body after methyl methacrylate cement has hardened and the catheter 60 being removed. When hardened at the exact location of the compression fracture, the capacity of the wedge shaped cement mold 68 and its vertebral body to withstand future weight is now increased.

The general guideline in the prior art says that the geometry of the balloon should take into account that at least 40% of the cancellous bone volume needs to be compacted if there is fracture or the loss of cancellous bone mass from osteoporosis. From the prior art, compaction can be from a low of 30% to a high of 90%. It is known that compacting less cancellous bone volume (30-40%) can leave too much diseased cancellous bone behind where it remains weak and can later collapse. On the other hand, compacting too much bone (75-90%) can leave much of the targeted fractured bone displaced, fractured or depressed. The optimal expansion of the vertebral body that can elevate or push the fractured cortical wall back to or nears its normal anatomic position appears to be around 50-75%. As FIG. 7 illustrates, a wedge shaped balloon will preferably compact at least 50% of the cancellous bone and will elevate or push only that cortical bone which is damaged. When such a cavity is created, the heel side of the wedge shaped cavity extends from the two end plates of the vertebral body thereby providing optimal support to the damaged side.

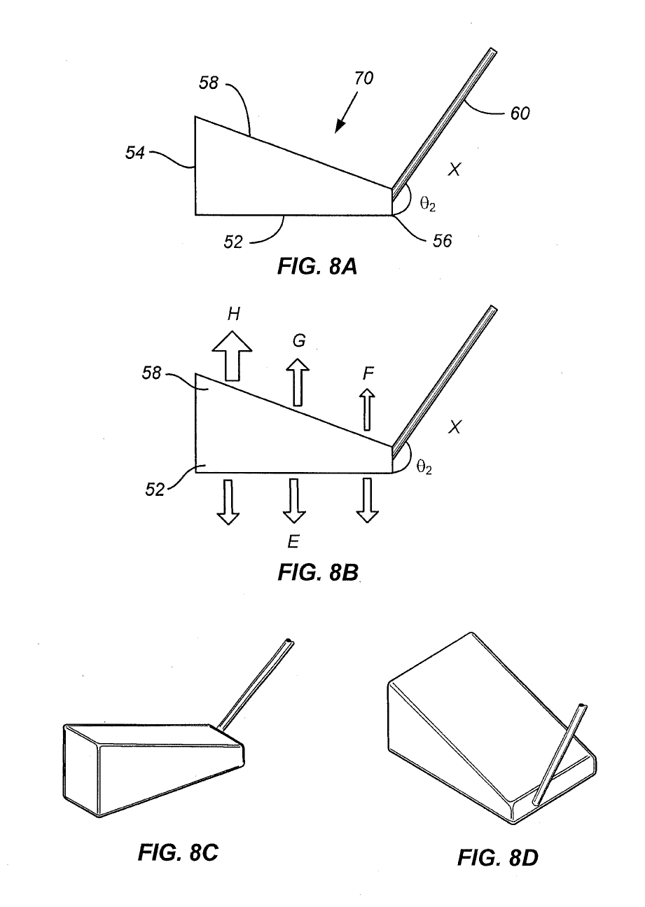

FIG. 8A shows another embodiment of the wedge shaped balloon 70 of the present invention in an inflated condition. In this embodiment, the heel 54 of the wedge shaped balloon is distal to the catheter tube 60. As such, the catheter tube 60 is attached to the toe side 56. In this embodiment, the catheter tube 60 is offset at a greater angle Ø₂ from the axis of the wedge shaped balloon 70. In this arrangement, the catheter tube 60 can be in the same axis with the pedicles 24 (FIGS. 1 & 2) thereby making the more preferable transpedicular approach possible. Although the catheter tube may be symmetrical and at a similar angle to the pedicles, when expanded, the base 52, heel 56 and toe 54 sides of the wedge shaped balloon can be parallel or perpendicular with the mid-lateral axis 6 and mid-anterior-posterior axis 8 (see, FIGS. 1 & 2) of the vertebral body, respectively.

Again, the advantages of a wedge shaped balloon are shown in FIG. 8B by employing the mechanics of a wedge. As mentioned earlier, a wedge functions by converting a force E applied from its base side 52 to its inclined side 58. The mechanical advantage (MA) of the wedge in this inverted embodiment from FIG. 6B is still the ratio of the length of its slope (S) to its width (W) or MA=S/W. The more acute, or narrow, the angle of a wedge, the greater the ratio of the length of its slope to its width, and thus the mechanical advantage it will yield F<G<H or H>G>F.

FIGS. 8C & D shows several different toe side embodiments of the wedge shaped balloon. Various sizes of the wedge shaped balloon are shown in FIGS. 8C & D. Again, such different shapes allow the physician to select a geometry that correctly matches the fracture based on diagnostic images (i.e. MRJ, CRT). Furthermore, the offset catheter offers surgeons the choice of either the left or right pedicle (e.g. FIG. 8D).

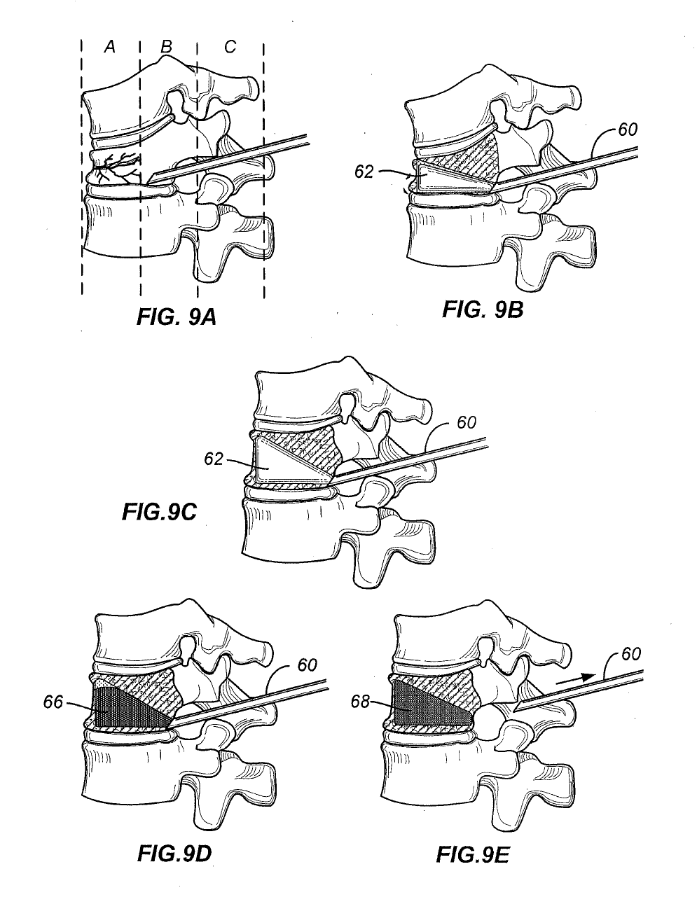

Referring to FIGS. 9A-E, FIG. 9A is a schematic side view of a lumbar vertebra showing anterior compression break A in a substantially collapsed condition with a catheter deployed by the posterolateral access. FIG. 9B shows how the partial inflation of the wedge shaped balloon 62 uses the mechanics of a wedge to shift the compression forces toward the anterior compression break A. FIG. 9B also shows how the offset catheter can be used for the posterolateral approach and is not exclusive to the transpedicular approach. In this case, it may be preferable to enter the vertebral body between the inferior and superior end plates of the vertebral body. With this procedure, it is easier to reach the posterior quarter of the vertebral body because the higher catheter angles employed may not be possible with the transpedicular approach. This illustrates the versatility of the present invention. In particular, the present invention allows variable access to the vertebral body (e.g., either the transpedicular or the posterolateral approach). The variable angle achieved by the offset catheter gives the surgeon more flexibility in choosing the proper balloon geometry for the different compression breaks.

FIG. 9C shows a fully inflated balloon 62 restoring the original height of the compressed vertebra. FIG. 9D is similar to FIG. 9C but shows how removing the balloon leaves behind a wedge shaped cavity 66 that is being injected with methyl methacrylate cement. Such a wedge shaped cavity 66, when filled with bone cement, can provide optimal support to the area of the compression injury, especially if such fractures are in the anterior column A (FIG. 9A). FIG. 9E is a similar view to FIG. 9D but shows the vertebral body after methyl methacrylate cement has hardened and the catheter 60 is removed. When hardened at the exact location of the compression fracture, the capacity of the vertebral body to withstand future weight is now increased from the support provided by the heel side of the wedge shaped cement mold 68.

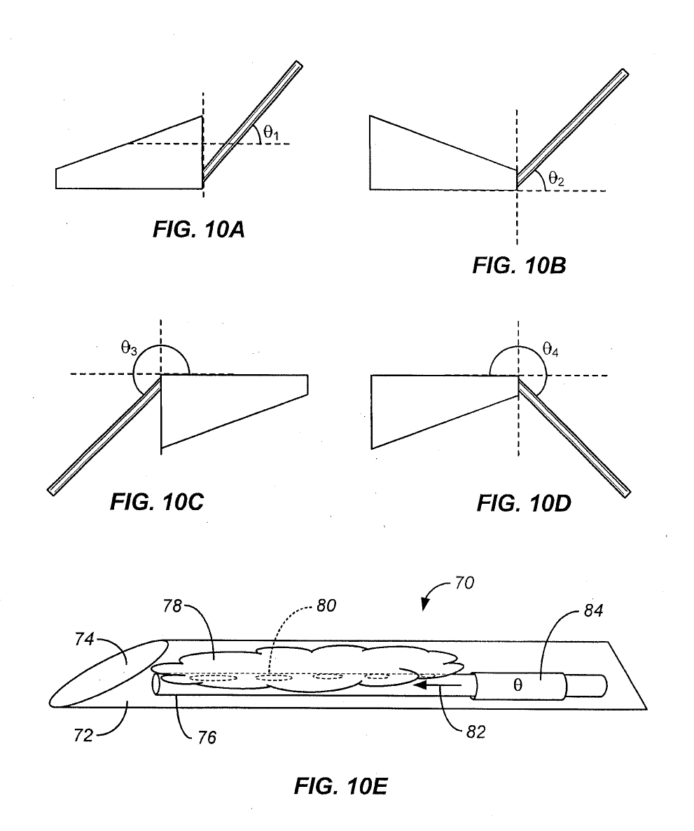

FIG. 10A-C shows the varying ways possible in the present invention to connect the catheter to the wedge shaped balloon in the heel (FIG. 10A), toe (FIG. 10B), inverted heel (FIG. 10C) or inverted toe (10D) configurations. In these configurations, the degree of connection can range from as low as 30-45° (see, Ø_{1 & 2}) to greater than 90° (see, Ø_{3 & 4}). Such offset angles are not possible with the prior and existing symmetrical balloon catheters, especially those that are aligned along the same medial axis. Since these offset angles were not possible with the symmetrical catheters, any shaped balloon symmetrical with its catheter is likely to be inappropriately placed within the vertebral body.

FIG. 10E shows an embodiment of the present invention that generates virtually any desired offset geometry. As shown in FIG. 10E, the catheter tube assembly 70 of the present invention can include an outer tube 72, that is preferably made of a rigid metal, and an inner catheter tube 76. More rigid materials have greater stiffness essential in the manipulation of bone catheters and may include stainless steel or nickel-titanium alloys. The outer catheter tube 72 is also preferably beveled 74. Within the outer tube 72 is the inner catheter tube 76 that is secured and enclosed by the outer tube 72. Unlike the prior art (FIG. 4), the distal end of the inner catheter tube 76 preferably remains inside the outer catheter tube 72. The diameter of the inner catheter tube 76 is preferably much smaller than the outer catheter tube 72. Unlike the prior art, the balloon 78 is preferably not bonded to the distal end of the inner catheter tube 76, but to the sides of the inner catheter tube 76. On the top of the inner catheter tube 76 is a row of various sized apertures 80 in this embodiment that allow an inflation medium 82 (e.g., air) to flow into the balloon through a flow passage of the hollow inner catheter tube thereby causing expansion of a wedge shaped balloon 78. In the preferred embodiment, the inner catheter tube 76 comprises a section 84 or may be entirely made of a shape memory alloy (SMA). Such a shape memory alloy permits the inner catheter tube 76 to generate any desired offset geometry and orient the wedge shaped balloon to the particular geometry of the targeted bone. When properly oriented, the wedge shaped balloon can employ the superior mechanics of the wedge to decompress spinal fractures of the vertebral body.

Shaped memory alloys remember its original, cold-forge shape and returns to that shape after being deformed by applying heat (i.e., body heat). Preferred shaped memory alloys include copper-zinc, aluminum-nickel, copper-aluminum-nickel or nickel-titanium (NiTi) alloys. NiTi alloys for example can have their transition temperature set below the expected room temperature. This allows the metal to be deformed under stress, yet retain its intended shape once the metal is unloaded again. The inner catheter tube 76 in FIG. 10E may be entirely made of SMA or just a section 84. This section 84 or the entire inner catheter tube can be deformed to virtually any angle to the desired offset geometry of the targeted bone. Such a deformity can be set at predetermined angle (e.g., Ø₁, Ø₂, Ø₃, Ø₄, as illustrated in FIG. 10) so that when the inner catheter 76 exits the outer catheter tube 72 it snaps into its intended shape and angle. The present invention allows the catheter to achieve angles that were not possible with previous symmetrical catheter tubes. When substantially expanded (as shown in FIGS. 7 & 9), the wedge shaped balloon is now offset from outer catheter tube 72 axis, so that its base 52 (FIGS. 6 & 8), if desired, can be parallel with the mid-anterior-posterior axis 8 and its heel 54 and toe 56 are parallel with the mid-lateral axis 6 (FIGS. 1 & 2) of the vertebral body. Once in this configuration, the superior mechanics of the wedge provided by the wedge shaped balloon may be employed.

The bevel 74 at the distal end of the catheter tube assembly 70 shown in FIG. 10E has many uses. Since the bevel end can be cut at any angle, it can be made into either a blunt or a sharp 74 cutting device as it enters the cancellous bone. As such, it may act as a cutting device to cut or slice through cancellous bone. It can also act as a probe to determine the difference of whether the catheter is passing through cancellous or cortical bone. In one embodiment of the present invention, the bevel 74 cuts through cancellous bone until such time when it hits cortical bone. Upon reaching cortical bone, the bevel 74 can act as a probe as it cuts and finds the cortical bone. Additionally, it can anchor itself to cortical bone of the vertebral body to secure the catheter tube assembly 70 to either the inferior or superior end plates. Furthermore, the bevel provides an acute aperture for the inner catheter tube 76 to exit from the outer catheter tube 72. As it exits the outer catheter tube 72, the inner catheter tube can then assume its predetermined angle generated by the shape memory section 84. Since the bevel can be cut at any angle, the bevel 74 can also provide a stop plate for inner catheter tube 76 as it exits the outer tube 72 given that the outer tube 72 is also at an acute angle. In combination with the SMA section, the bevel 74 stops overextension of the inner tube 76 as it snaps into its predetermined angle. With both mechanisms, the base 52 of the prism-like balloon can now be aligned and parallel with either the end plate of the inferior or superior end plate of the vertebral body. The bevel 74 edge of the present invention may eliminate need for many of the prior art catheter instruments used previously to cut, probe and secure catheters.

Wedge shaped balloon 78 is preferably made of a flexible, yet durable material, such as polyethylene tetraphthalate (PET), Kevlar or other strong polymeric materials, that can be inflated to pre-determined shape. These durable materials help the balloon maintain a desired shape and size. By contrast, thin elastic latex balloons are not preferred because they can assume unpredictable spherical shapes that are dangerous, especially when the spinal cord is nearby. Preferably, the wedge shape chosen corresponds to the morphology of the targeted bone. This can be determined using diagnostic imaging equipment, such as plain films, spinous process percussion, MRI or CRT scanning. The use of flexible, yet durable materials also permits the application of appropriate and targeted application of pressure to a defined geometry to accurately compress cancellous bone.

Another feature of the catheter tube assembly 70 shown in FIG. 10E are the apertures 80 on top of the inner catheter tube 76. The inner catheter tube 76 contains various sized apertures 80 along its top edge. In turn, the balloon 78 surrounds and are bonded around such apertures 80. Since the inner catheter tube 76 is a hollow spherical channel, it permits inflating air or liquid under pressure to enter and exit the inner catheter tube at different flow rates to inflate the balloon. When so inflated, the balloon begins to exhibit wedge mechanics during inflation, especially when fully inflated. The larger sized apertures 80 are preferably oriented to the base 52 of the wedge shaped balloon. As the slope of the incline side 58 slides downward, the apertures preferably decrease in size to match the flow of the inflating liquid with the volume of the wedge shaped balloon.

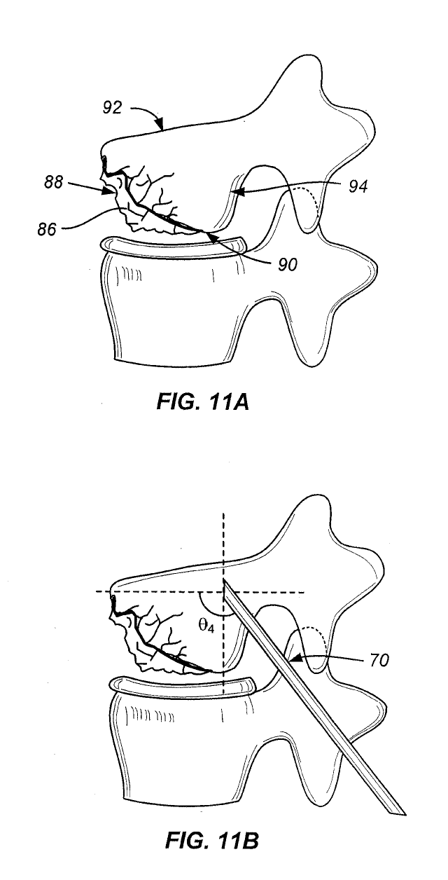

The preferred method of the present invention is shown in FIGS. 11-13. In FIG. 11A, an acute severe compression fracture 86 has occurred in both the anterior vertical 88 and inferior 90 cortical plates. As shown in FIG. 11A, a considerable amount of the cortical bone is displaced, fractured or depressed. In this case, the geometry of the balloon should take into account that at least 40% of the cancellous bone volume needs to be pushed outward. Pushing less cancellous bone volume will leave too much diseased cancellous bone behind where it remains weak and can later collapse. If too much is pushed, adjacent and healthy cortical bone wall of the vertebral body may fracture.

This case is further complicated by the fact that only the superior plate 92 and posterior plate 94 of the vertebral body remain intact. To use both the superior 92 and posterior 94 plates as supports, the wedge shaped balloon of the present invention should be inverted (FIG. 10C) to push or compact cancellous bone toward the inferior plate 90 (downward) and anterior plate 88 (backwards). Although a transpedicular approach is possible, a catheter tube assembly 70 of the present invention preferably enters between the inferior 90 and superior 92 end plates of the body and the posterior quarter of vertebral body shown in FIG. 11B at an extreme angle Ø₄ greater than 90 degrees.

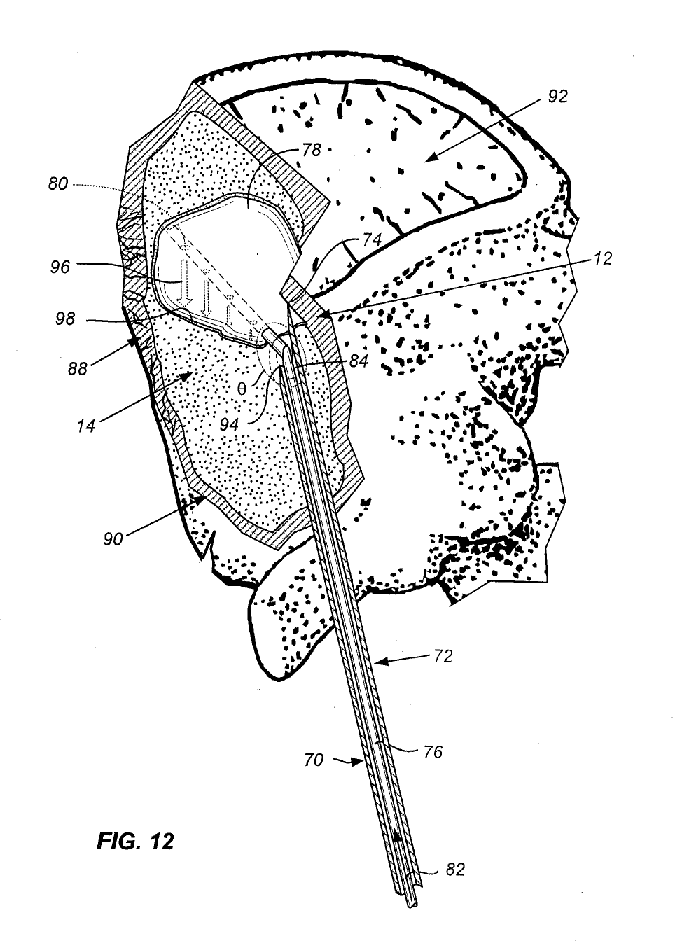

Once entry into the vertebral body is made, the next method step of the present invention shown in FIG. 12 is to insert the catheter tube body assembly 70 into the vertebral body. As it does so, its bevel 74 edge slices through the interior of the vertebral body. The destination of the bevel 74 edge is the junction between the superior and posterior plates where the bevel 74 (outer catheter tube 72) preferably imbeds itself into the cortical bone 12 to lock itself into the wall of the superior wall plate 92 for stabilization. When the bevel 74 makes contact with the cortical bone 12 of superior wall plate 92 of the vertebral body, the inner catheter tube 76 is pushed through the outer catheter tube 72 until it reaches the SMA section 84. Upon sliding beyond the outer catheter 72 and bevel 74, the inner catheter tube 76 snaps to its predetermined angle Ø. To prevent over extension, the bevel edge 94 acts as a stop plate for inner catheter tube 76 as it exits the outer tube 72. The inner catheter tube 76 and the base 52 of the wedge shaped balloon 78 preferably becomes parallel with the natural mid-anterior-posterior axis 8 (FIGS. 1 & 2) and thus parallel with the end plate of the superior wall plate 92 of the vertebral body. With the support of the superior end plate 92, the balloon 78 is ready to be expanded and begin compressing the cancellous bone 14 toward the damaged inferior 90 and anterior 88 plates.

The shape and size of the balloon 78 shown in FIG. 12 is preferably determined by a pre-operative diagnostics. In this case, an inverted wedge shaped balloon 78 of the present invention with its catheter on the toe side is chosen to take advantage of the wedge mechanics toward the inferior 90 and anterior 88 plates. Upon inflation, wedge forces can be brought to bear on the fractured corner of the inferior 90 and anterior 88 end plates with less force toward the remaining cortical bone edges. The balloon is gradually injected with inflating medium 82 through the inner catheter tube 76. Equal volume flow is preferably achieved through the various sized apertures 80 on the inner catheter tube 76 so that all sides of the wedge shaped balloon inflate equally throughout the vertebral body. When the balloon begins to fill, the wedge pressure 96 is now directed toward the anterior-inferior plate juncture. If a cylindrical balloon was used, such a targeted application of force would not be possible and would create an unequal distribution of compressed cancellous bone throughout the vertebral body. As the balloon reaches the anterior-inferior plate juncture or the balloon becomes fully expanded, the inflation is stopped signaling the termination of the cavity 98 preparations.

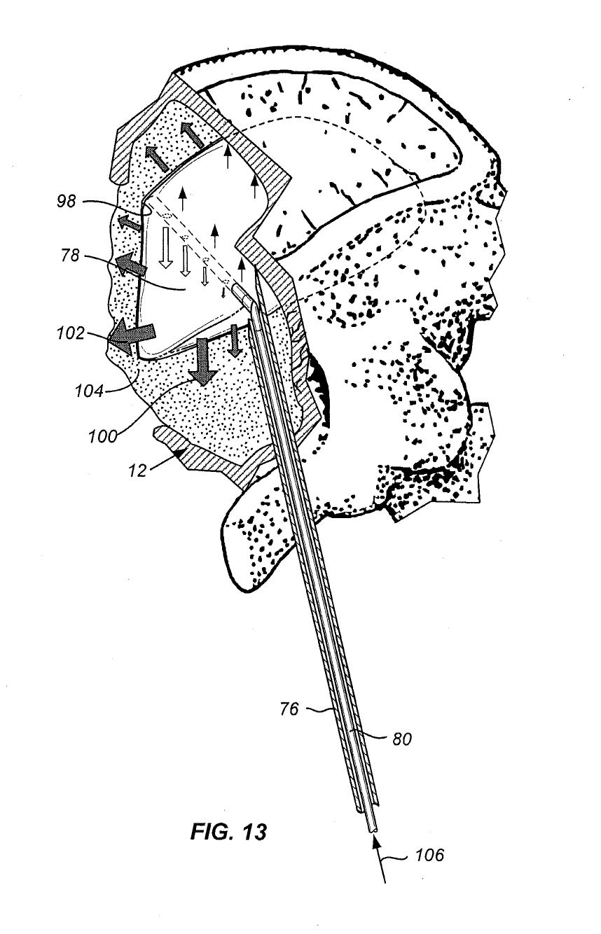

As shown in FIG. 13, when the balloon inflates it forces cancellous bone laterally 100 and outwardly 102 toward the fractured vertebral body. This compacts the cancellous bone 14 and creates a cavity 98 in the interior of the vertebral body. The compacted bone forms a dam 104 internal to the anterior 88 and inferior 90 plates so as to block any further fracture of the vertebral body. After the balloon 78 is deflated, the inner catheter tube 80 and balloon 78 are removed from the outer catheter tube 72. The outer catheter tube 72 can also to used to clean out any effluent in the cavity. After irrigation, artificial bone substitute that may include synthetic bone or methyl methacrylate cement 106 is injected into the cavity.

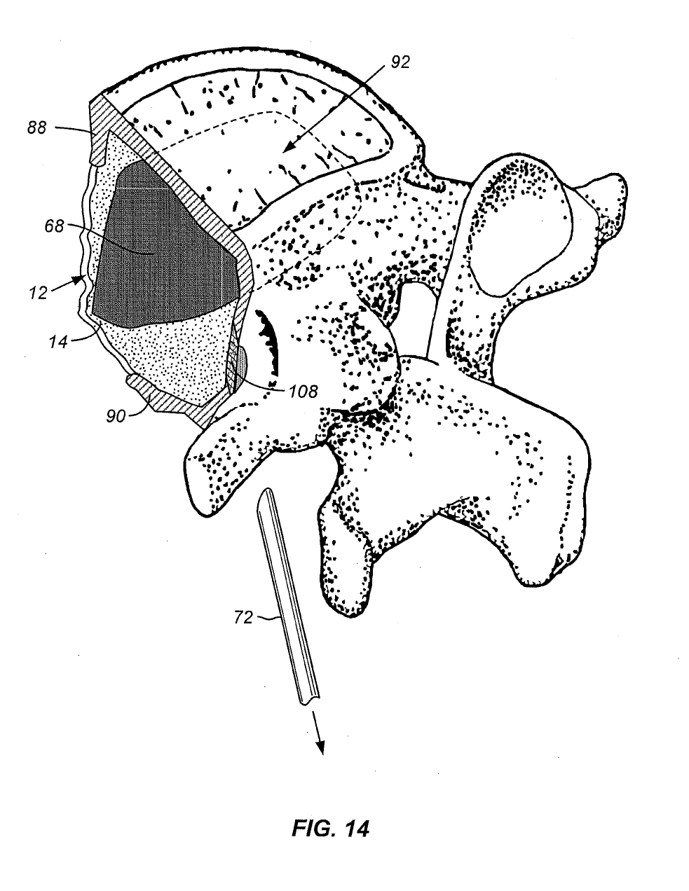

As shown in FIG. 14, when the proper volume of bone cement is reached, the outer catheter tube 72 is removed to let the bone cement to harden and fill the entry hole 108. The present invention closely restores the vertebral body to its normal anatomical height and in doing so corrects the spinal angular deformity caused by a fracture or disease. It also leaves behind a cement mold 68 so that the capacity of the vertebral body to withstand future weight is increased. The position of the cement mold 68 is so optimal that cortical bone 12 at the anterior 88 and inferior 90 plates of the vertebral body are not damaged or further fractured giving the cancellous bone 14 an opportunity over time to restore or replace the fractured cortical bone 12. During this time, the cement mold 68 reaching from the one end of the inferior end plate 90 to the superior end plate 92 optimally supports and restores the vertebral body to its original anatomic height and corrects the spinal angular deformity caused by a fracture or disease.

In the foregoing specifications, the invention has been described with reference to specific preferred embodiments and methods. It will, however, be evident to those of skill in the art that various modifications and changes may be made without departing from the broader spirit and scope of the invention. For example, while a wedge shaped balloon provides superior mechanics and optimal support, those of skill in the art will recognize that alternative generally triangular or rectangular prism-like shapes also be used as in the methods case described above. Various different shape memory alloys and variations of such to the catheter tube assembly can further improve the delivery of the spinal fixation device. Accordingly, the specification and drawings are to be regarded in an illustrative, rather than restrictive, sense; the invention being limited only by the appended claims.

What is claimed is:

1. A medical device for restoring compressed spinal vertebra comprising a generally wedge shaped balloon fixed to a catheter tube assembly.

2. The medical device of claim 1 wherein said generally wedge shaped balloon is either a triangular or rectangular prism.

3. The medical device of claim 1 wherein said generally wedge shaped balloon has heel, toe and incline sides.

4. The medical device of claim 1 wherein said generally wedge shaped balloon is made of material selected from a group consisting of polyethylene tetraphthalate (PET) and Kevlar.

5. A medical device for restoring compressed spinal vertebra comprising a catheter tube assembly consisting of an outer catheter tube and a concentric inner tube with a generally wedge shaped balloon affixed to said inner catheter tube.

6. The medical device of claim 5 wherein said outer catheter tube is made of rigid material selected from a group consisting of stainless steel or nickel-titanium alloys.

7. The medical device of claim 5 wherein said outer catheter tube is beveled at its distal end.

8. The medical device of claim 5 wherein said inner tube is made from one or more shape memory alloys selected from a group consisting of copper-zinc, aluminum-nickel, copper-aluminum-nickel or nickel-titanium (NiTi) alloy.

9. The medical device of claim 5 wherein said inner catheter tube contains apertures at its distal end.

10. A method for restoring a damaged vertebra comprising the steps of:
creating an entry hole to access said damaged portion of said vertebra;
inserting a catheter tube assembly through said entry hole, wherein said catheter tube assembly comprises an outer catheter tube and a concentric inner catheter tube with a generally wedge shaped balloon attached to it;
inflating said generally wedge shaped balloon within said damaged portion of said vertebra to create a generally wedge shaped cavity;
deflating said wedge shaped balloon after said generally wedge shaped cavity has been created in said vertebra and removing said balloon from said vertebra;
inserting bone cement through said outer catheter tube to fill said generally wedge shaped cavity and allowing said bone cement to harden.
